(19) United States
(12) Patent Application Publication
(10) Pub. No.: US 2023/0154572 A1
(43) Pub. Date: May 18, 2023

(54) RETROSYNTHESIS-RELATED SYNTHETIC ACCESSIBILITY

(71) Applicant: INSILICO MEDICINE IP LIMITED, Hong Kong (HK)

(72) Inventors: Bogdan ZAGRIBELNYY, Pak Shek Kok (HK); Evgeny Olegovich PUTIN, Saint Petersburg (RU); Sergei Andreevich FEDORCHENKO, Perm (RU); Yan A. IVANENKOV, Moscow (RU); Aleksandrs ZAVORONKOVS, Pak Shek Kok (HK)

(21) Appl. No.: 17/911,376

(22) PCT Filed: May 11, 2021
(86) PCT No.: PCT/IB2021/054029

§ 371 Date: Sep. 13, 2022

Related U.S. Application Data
(60) Provisional application No. 63/025,135, filed on May 14, 2020.

(51) Int. Cl. G16C 20/30; G16C 20/10; G16C 20/70

(52) U.S. Cl. CPC G16C 20/30; G16C 20/10; G16C 20/70

(57) ABSTRACT

A method for training model to calculate synthetic accessibility includes: accessing molecule database and obtaining molecule; virtually slicing the molecule into fragments; determining a fragment frequency of fragments; calculating molecular descriptors for the fragments; calculating synthetic difficulty score for the molecule; and storing the synthetic difficulty score in a database. A method of evaluating molecular synthetic accessibility includes: selecting target molecule; decomposing the target molecule into molecular fragments; calculating a synthetic difficulty score for the molecular fragments for the target molecule; determining a sum of synthetic difficulty scores for the molecular fragments; determining a fragment density of the molecular fragments; calculating the synthetic accessibility score from the sum of synthetic difficulty scores and fragment densities; and providing the synthetic accessibility score for the target molecule.

### CROSS-REFERENCE TO RELATED APPLICATIONS

This patent application claims priority to U.S. Provisional Application No. 63/025,135 filed May 14, 2020, which provisional is incorporated herein by specific reference in its entirety.

### BACKGROUND

Chemical synthesis planning is an integrative, complex, long and resource-consuming process in the modern drug design and development (DDD) industry. It includes a lot of subtasks such as: synthetic accessibility estimation, manual creation or machine-based prediction of relevant synthetic path frequently using computer-aided approaches, the assessment of available on the market starting building blocks and ready-to-use reactants, and the selection of correct reaction properties (solvents, catalysts, base, temperature, pressure).

Big pharmaceutical companies synthesize molecules on a large scale. In part, this may be a reason that one of the most crucial steps in chemical synthesis planning is the estimation of synthetic accessibility (SA) for compounds. In general, SA measures the feasibility of synthesis in terms of many medicinal chemistry-based and market-based metrics. Therefore, often SA represents some value or score for considering a route for a compound to be synthesized. Such scoring procedure of SA is very useful, because it allows to prioritize synthesis, save actives and time along with fitting into the desired hit rate of generation. It should be noted that there is no standard definition of SA and thus every pharma or biotech company creates its own original computer-aided method to estimate and validate SA. Such methods can take into account different aspects of synthesis, namely the amount of complex substructures in the resulting compound, in-house available building blocks and reactants in vendor's databases as well as financial benefits in their usage, the number of stages in the predicted synthetic paths, and the like.

Recently, there has been success in the field of DDD and, in particular, in chemical synthesis planning. Therefore, a modern understanding of SA can be conditionally represented by two commonly used groups of methods: (1) molecular descriptor-based where molecular descriptor (MD) is a characteristic of a molecule like molecular weight, carbon atoms count; or (2) membrane permeability and data-driven approaches. The most notable and commonly used descriptor-based method is SA Score. SA Score is solely based on molecular descriptors, and it calculates the subtraction of two scores. The first one depicts historical synthetic knowledge by analyzing common structural features of molecule fragments (e.g., fragment means a substructure of a molecule acquired by fracturing molecule by available retro-synthetic connections and a molecule without available retro-synthetic connections cannot be split and thus only contains itself as a fragment) in a prepared database of already synthesized molecules. The second subtracting score works like a penalty, and is a number that characterizes the presence of complex structural features in the considered molecules. As a result, SA Score shows a compromise between fast complexity-based, and resource-intensive full retrosynthetic approaches.

On the other side, data-driven approaches such as synthetic complexity score (SC Score, SYBA, RAscore) are not dependent on hand-crafted features of molecules and thus is more robust and objective. Because such methods do not rely on chemical intuition about synthetic complexity of compounds they are independent in terms of concrete molecular design problems and can be more seamlessly transferred from one synthesis planning task to another.

Aforementioned SC Score is a perceptible example of data-driven approaches, which use precedent chemical reaction knowledge to learn a function approximator for the evaluation of synthetic complexity of compounds. As a function approximator SC Score uses a fully-connected artificial neural network (ANN), which is trained with standard backpropagation algorithms on a large database of known synthesizable drug-like molecules with their known synthetic paths. The key idea behind SC Score is to learn such a ranking function that should be greater of the reaction's product than of any distinct reactants in this reaction. Thus, SC Score does not account for decomposition or single and double replacement chemical reactions. Because the method is fully data-driven, and it pushes the mentioned ranking system to be satisfied for any given training reaction, it also can fail on the testing stage in particular cases where a complex molecule is presented only as a reactant but not as a product.

The original SC Score uses molecular fingerprints as a characteristic of chemical reaction to train the model. However, chemical reactions can be represented in a string-based format. The simplified molecular-input line-entry system (SMILES) is a specification in the form of a line notation for describing the structure of chemical species using short ASCII strings. Fragments of a molecule are also valid SMILES with special symbols for connectivity information. A molecule always contains all its fragments, which can be linked into the whole molecule again. SMILES strings can be imported by most molecular editors for conversion back into two-dimensional drawings or three-dimensional objects of the molecules.

Another approach referred to as SYBA (SYnthetic Bayesian Accessibility) is a fragment-based method for the distinguishing between easy- (ES) and hard-to-synthesize (HS) compounds. It is based on a Bernoulli naïve Bayes classifier that is used to score contributions to individual fragments based on their frequencies in the database. SYBA was trained on ES molecules available in the ZINC15 database and on HS molecules generated and filtered for complex compounds only.

Some of the algorithms are based not only on molecules, but on synthetic routes for novel compounds. AiZynthFinder is an example of such software that can be readily used in retrosynthetic planning. The algorithm is based on a Monte Carlo tree search that recursively breaks down a molecule to purchasable precursors. The tree search is guided by an artificial neural network policy that suggests possible precursors by utilizing a library of known reaction templates.

RAscore is a classifier trained on the retrosynthetic predictions of AiZynthFinder using the solved or unsolved labels based on vendor database of known compounds. The compounds were subsequently subjected to retrosynthetic analysis using AiZynthFinder, and labelled as solved or unsolved.

PostEra score is a retrosynthesis engine, which computes a synthetic accessibility score based on the routes found by AiZynthFinder, with a scoring function that balances several factors, including the cost/lead-time of the building blocks and how likely model deems the reactions to proceed. If multiple routes are found, which is the typical case, then the score is discounted based on the viability and diversity of backup alternative routes.

### SUMMARY

In some embodiments, a method for training a model to calculate synthetic accessibility can include: accessing a molecule database and obtaining a target molecule; virtually slicing the target molecule into molecular fragments; determining a fragment frequency of a plurality of molecular fragments of the target molecule; calculating molecular descriptors for the molecular fragments; calculating a synthetic difficulty score for the target molecule; and storing the synthetic difficulty score for the target molecule in a database having a plurality of synthetic difficulty scores for a plurality of molecules. In some aspects, the method can include receiving a training dataset of training molecules to obtain data of a chemical structure and properties of the target molecule. In some aspects, the slicing includes decomposing the target molecule to obtain synthesizable fragments, where a decomposition function: produces valid drug-like molecular structures; and is invertible so that obtained synthesizable fragments can be converted back to the target molecule. In some aspects, the decomposing is performed by a retrosynthesis-related decomposing function.

In some embodiments, the training method includes evaluating chemical properties of the synthesizable fragments. In some aspects, the evaluating is performed by calculation and aggregation of the molecular descriptors. In some aspects, the aggregation of molecular descriptors includes: Chiral Carbons Count, which is the number of chiral carbon atoms; Ring Count, which is the total number of rings; Ring Side Chains Count, which is the number of side chains attached to the ring systems; Spiro Count, which is the number of spiro carbon atoms; Biggest Ring Size, which is the number of atoms in the largest ring of molecular structure if it is bigger than 6, otherwise 0; Fused Rings Count, is the number of fused rings in a molecular structure; and Bridge Atoms Count, is the number of bridgehead atoms in the bicyclic pattern(s) of molecular structure.

In some embodiments, the determining of the fragment frequency is performed by applying a function of identity or logarithm to the number of molecules that contain the molecular fragment divided by the number of molecules in the training dataset.

In some embodiments, the computing of the fragment density function for the target molecule across the training dataset of training molecules is based on the frequencies of the synthesizable fragments in the training molecules.

In some embodiments, the training method includes aggregating fragment information of synthesizable fragments of the target molecule into fragment scores by taking the fragment frequencies into account. In some aspects, the aggregating is performed by a mathematical function applied to molecular descriptors of fragments and fragment frequencies. The method can include obtaining the fragment scores and saving the fragment scores in a database of fragment scores.

In some embodiments, the training method can include calculating the synthetic difficulty score as a product between a fragment density function and a linear combination of fragment scores and fragment frequencies. In some aspects, the method includes providing the calculated synthetic difficulty score as a synthetic accessibility score. In some embodiments, the training method includes normalizing the synthetic accessibility score to a desired scale with a mathematical function.

In some embodiments, a method of evaluating molecular synthetic accessibility can include: selecting a target molecule; decomposing the target molecule into molecular fragments; calculating a synthetic difficulty score for the molecular fragments for the target molecule; determining a sum of synthetic difficulty scores for the molecular fragments; determining a fragment density of the molecular fragments; calculating the synthetic accessibility score from the sum of synthetic difficulty scores and fragment densities; and provide the synthetic accessibility score for the target molecule.

In some embodiments, the method for determining synthetic accessibility includes obtaining data of chemical structure and properties of the target molecule. In some aspects, the method includes obtaining scores of synthesizable fragments from a trained model for calculating synthetic accessibility. In some aspects, the method includes calculating molecular properties for fragments whose properties cannot be obtained from the trained model. In some aspects, the method includes calculating fragment density functions for fragments whose fragment density functions cannot be obtained from the trained model. In some aspects, the method includes comprising aggregating processed information to the synthetic accessibility score of the target molecule. In some aspects, the decomposing is performed by a retrosynthesis-related decomposing function, optionally selected from open-sourced BRICS or RECAP algorithms.

In some embodiments, the method for determining synthetic accessibility includes evaluating chemical properties of the synthesizable fragments. In some aspects, the evaluating is performed by calculation and aggregation of the molecular descriptors, such as those described herein (e.g., same as in the training methods). In some aspects, the method includes computing a fragment density function for the target molecule across the training dataset of training molecules based on the frequencies of the synthesizable fragments in the training molecules. In some aspects, the method includes aggregating processed information of synthesizable fragments of the target molecule into fragment scores by taking the fragment frequencies into account. In some aspects, the aggregating is performed by a mathematical function applied to molecular descriptors of fragments and fragment frequencies. In some aspects, the synthetic accessibility score are scaled from one to n, where n>1. In some aspects, a vendor database for the target molecule or synthesizable fragments is not present.

In some embodiments, the method for determining synthetic accessibility can include: calculating a synthetic difficulty score for the target molecule by an iterative protocol including: identifying all molecular fragments of the target molecule; checking for all molecular fragments in a synthetic difficulty score database; when a molecular fragment is the synthetic difficulty score database, add the synthetic difficulty score for the molecular fragment to an array of synthetic difficulty scores; when a molecular fragment is not in the synthetic difficulty score, then: calculate molecular descriptor for the molecular fragment; calculate the synthetic difficulty score for the fragment with a minimum frequency; and add the calculated synthetic difficulty score for the molecular fragment to an array of synthetic difficulty scores.

In some embodiments, one or more non-transitory computer readable media storing instructions that in response to being executed by one or more processors, cause a computer system to perform operations, the operations comprising the computer method of training a model to calculate synthetic accessibility in accordance to an embodiment.

In some embodiments, one or more non-transitory computer readable media storing instructions that in response to being executed by one or more processors, cause a computer system to perform operations, the operations comprising the computer method of evaluating molecular synthetic accessibility in accordance to an embodiment.

In some embodiments, a computer system can include: one or more processors; and one or more non-transitory computer readable media storing instructions that in response to being executed by the one or more processors, cause the computer system to perform operations, the operations comprising the computer method of training a model to calculate synthetic accessibility in accordance to an embodiment.

In some embodiments, a computer system can include: one or more processors; and one or more non-transitory computer readable media storing instructions that in response to being executed by the one or more processors, cause the computer system to perform operations, the operations comprising the computer method of evaluating molecular synthetic accessibility in accordance to an embodiment.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 includes a flow diagram illustrating a method of training a model to calculate synthetic difficulty score.

FIG. 2 includes a schematic diagram of a computing architecture that is configured for training a model to calculate synthetic difficulty score.

FIG. 3 includes a flow diagram illustrating a method of evaluating molecular synthetic accessibility.

FIG. 4 includes a schematic diagram of a computing architecture that is configured for training a model to evaluate molecular synthetic accessibility.

FIG. 5A includes a flow diagram illustrating a method of training a model to calculate synthetic accessibility.

FIG. 5B includes a schematic diagram of a computing architecture that is configured for training a model to calculate synthetic accessibility.

FIG. 6 includes a schematic diagram of a computing device that can perform the computing methods.

FIG. 7 includes a graph that shows dependency between two scoring engines.

FIG. 8 includes molecule structures and the SA and ReRSA graphs thereof, which show the dependency between the scores and the steps in the selected routes for the molecule.

FIG. 9 includes a graph that shows the mean score versus the number of molecules in the database, and shows the dependence of scores on the size of the training dataset.

FIGS. 10A-10C show representative examples of known bioactive compounds accompanied by the calculated ReRSA Scores.

FIG. 11 shows molecular structures and the calculated ReRSA Scores.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the proposed approach called retrosynthesis-related synthetic accessibility (ReRSA) estimation is a data processing protocol where the higher the occurrence (frequency) of “ready-to-synthesis fragments” in a molecule, the higher the synthetic accessibility of that molecule. The method can include a step to define what is a “ready-to-synthesis fragment” and/or identify those “ready-to-synthesis fragment” of a molecule to be synthesized. In the ReRSA method, a “ready-to-synthesis fragment” (RTSF) is a fragment that can be synthesized, which can be automatically obtained or identified by some predefined retrosynthesis-like decomposition procedure of molecules from a prepared virtual screening library of compounds, such as in a training dataset. Such a library should contain a large amount of already known synthetically accessible drug-like molecules. The best fit for that role are ready-to-use compound aggregators like open-sourced PubChem, ZINC and ChEMBL or vendor stocks like ChemDiv, Enamine or commercial databases such as Clarivate Analytics Integrity (Cortellis Drug Discovery Intelligence).

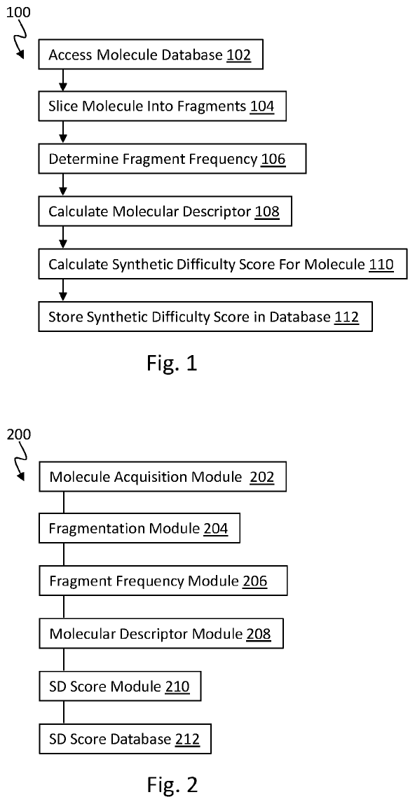

FIG. 1 illustrates a method 100 of data processing of molecule data to obtain a synthetic difficulty (SD) score (SD Score) for a target molecule. The method 100 can determine a plurality of different SD Scores for a single molecule when there are a plurality of different synthetic pathways. The SD Score can be used to determine whether or not a molecule should be synthesized based on its difficulty of synthesis or when the difficulty of synthesis (e.g., SD Score) is worse compared to those of other target molecules. For example, the better SD Score between two compounds with similar bioactivity can determine which compound becomes a lead for drug development. Also, the SD Scores for one or more molecules can be included in an SD Score Database. This database allows for the accession and use of SD Scores for molecular synthesis analysis.

The method 100 can obtain molecule data from a molecule database (block 102), such as a commercial database (e.g., from a vendor). The molecule data is then processed through a fragmentation protocol that slices the one or more molecule (e.g., all molecules) into molecular fragments (block 104), such as the RTSFs. The frequency of each molecular fragment (fragment frequency, “FF”) is then determined for the library of molecules in the database (block 106), which can provide an array of frequencies for the fragments. Here, the frequency of each fragment can be determined and stored in the database. Also, the fragment frequency can be associated with the molecule in the database. The molecular descriptor (MD) is calculated for every unique fragment in the molecule (block 108). The SD Score is then determined from the FF and MD (block 112) by aggregation thereof. The SD Score is stored in a SD Score Database (block 112) (e.g., dictionary of SD Scores). The SD Score Database can then be used for molecule synthesis analyses. In some aspects, the method 100 is a training method for a model. As such, the SD Score model is trained with the dataset in the method 100, which allows for a SD Score protocol to use the trained model along with the SD Score Database. This facilitates determining the ReRSA. In a summary, the method can include: Split molecules using predefined algorithm; Acquire frequencies from learned base; Calculate descriptors as shown herein; Calculate scores as shown herein; and Store resulting scores.

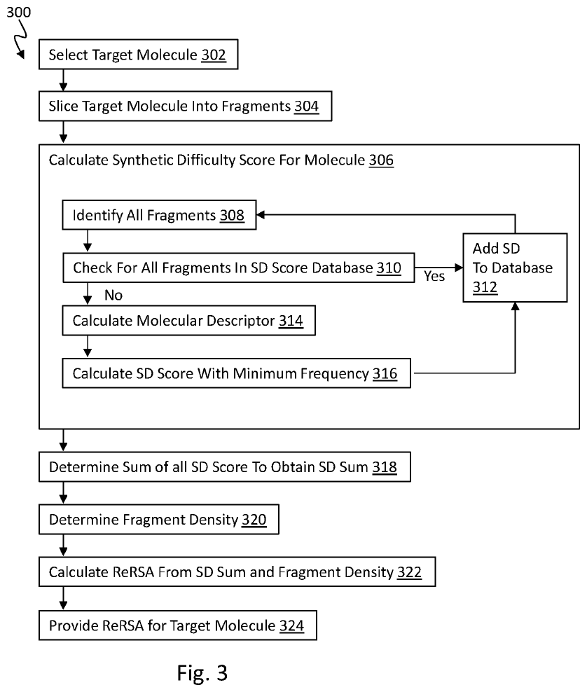

FIG. 2 illustrates an architecture 200 for performing data processing of the molecule data to obtain a synthetic difficulty (SD) score (SD Score) for a target molecule. The architecture 200 can include a molecule acquisition module 202 that is configured to obtain molecule data from a molecule database, such as a commercial database (e.g., from a vendor). The molecule data is then processed through a fragmentation module 202 that slices the molecule into molecular fragments, such as the RTSFs. The frequency of each molecular fragment (fragment frequency, “FF”) is then determined for the library of molecules in the database by a fragment frequency module 206. The molecular descriptor (MD) is calculated by the molecular descriptor module 208 for every unique fragment in the molecule. The SD Score is then determined by the SD Score module 210 from the FF and MD. The SD Score is then stored in a SD Score Database 212.

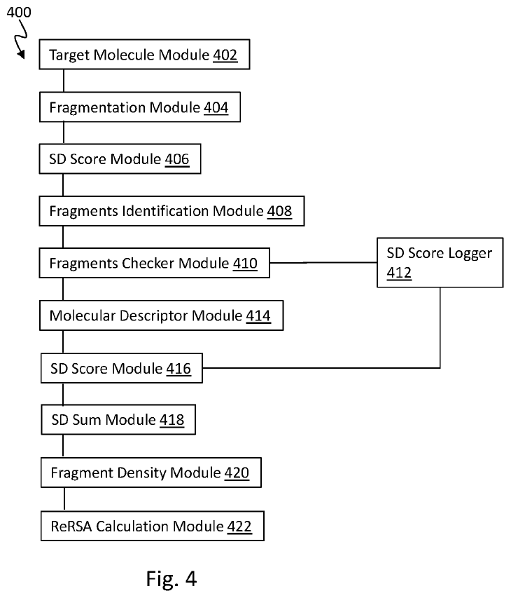

FIG. 3 illustrates a ReRSA method 300 that determines the ReRSA. The ReRSA method 300 includes obtaining a target molecule to score with ReRSA (block 302), where the molecule is in virtual format in descriptive data, such as graph data or string data. The target molecule is then split into molecular fragments (block 304). The molecular fragments are analyzed through an iterative SD Score operation (block 306). The iterative SD Score operation (block 306) is performed until the SD Score for all molecular fragments of the target molecule are obtained.

The SD Score operation (block 306) includes the following procedure. All fragments of the target molecule are identified (block 308). All of the identified fragments are checked for an SD Score in the SD Score Database (block 310). If it is determined that an identified fragment is in the SD Score database (e.g., a SD Score Library), then the SD Score of that identified fragment is added to an array of fragments for the target molecule (block 312), which can be a listing of the array of fragments in a database with data for the target molecule. If it is determined that the identified fragment is not in the SD Score database, then the molecular descriptors (MD) for the identified fragment is calculated (block 314). Then the SD Score is calculated with a minimum frequency (block 316).

Once the SD Score is obtained for each fragment of the target molecule, the sum of all of the SD Scores of the fragments is calculated to obtain the SD Sum (block 318). Then, the fragment density (FD) is calculated to measure the relative density of the synthesizable fragments that are in the molecule (block 320). The ReRSA is then calculated from the SD Sum and FD (block 322). The ReRSA is then provided for the target molecule (block 324). The ReRSA of the target molecule can be saved in a database (e.g., ReRSA database), which allows for the ReRSA values for different molecules to be compared. For example, when multiple target molecules may have similar bioactivity, the ReRSA values can be used to determine which target molecule to use as a lead. In part, easier and less expensive synthesis can be helpful for preparation and commercialization of target molecules.

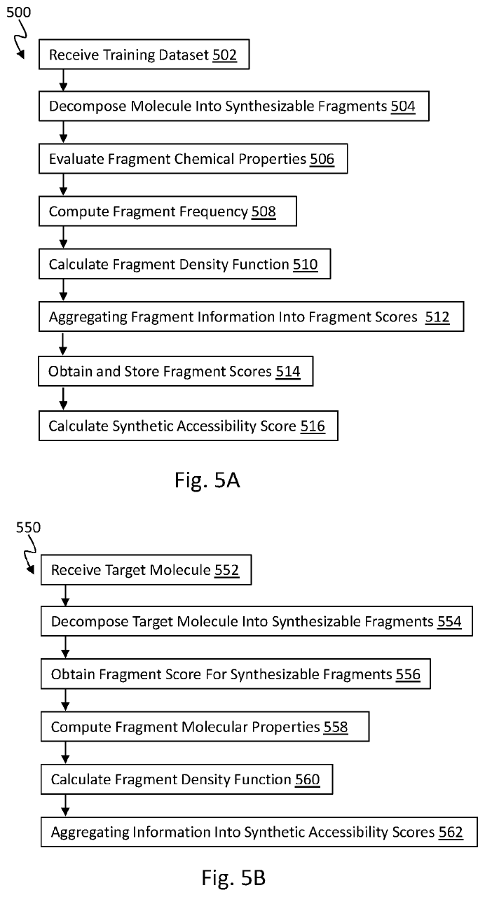

FIG. 4 illustrates a ReRSA architecture 400 that is configured to determine the ReRSA. The ReRSA architecture 400 includes a target molecule module that is configured for obtaining a target molecule to score with ReRSA, where the molecule is in virtual format in descriptive data, such as graph data or string data. A fragmentation module 404 is configured to split the target molecule into molecular fragments. A SD score module 405 is configured to perform operations so that the molecular fragments are analyzed through an iterative SD Score operation. The iterative SD Score operation is performed until the SD Score for all molecular fragments of the target molecule are obtained.

A fragment identification module 408 is configured so that all fragments of the target molecule are identified. All of the identified fragments are checked for an SD Score in the SD Score Database by a fragment checker module 410. If it is determined that an identified fragment is in the SD Score database (e.g., a SD Score Library), then the SD Score of that identified fragment is added to an array of fragments for the target molecule by a SD Score Logger 412. If it is determined that the identified fragment is not in the SD Score database, then the molecular descriptors (MD) for the identified fragment is calculated with a molecular descriptor module 414. A SD Score is calculated with a minimum frequency by a SD Score module 416. Once the SD Score is obtained for each fragment of the target molecule, the sum of all of the SD Scores of the fragments is calculated with an SD Sum module 418 to obtain the SD Sum. The fragment density (FD) is calculated with a fragment density module 420 to measure the relative density of the synthesizable fragments that are in the molecule. The ReRSA is then calculated from the SD Sum and FD by the ReRSA calculation module 422.

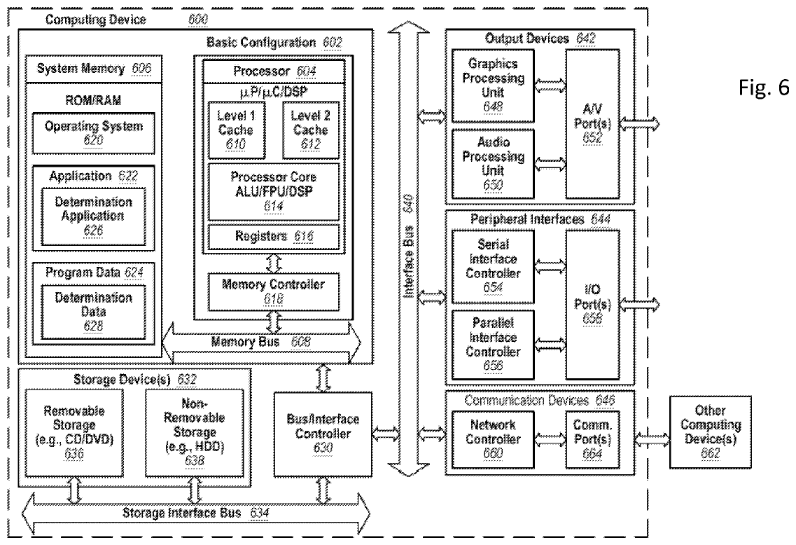

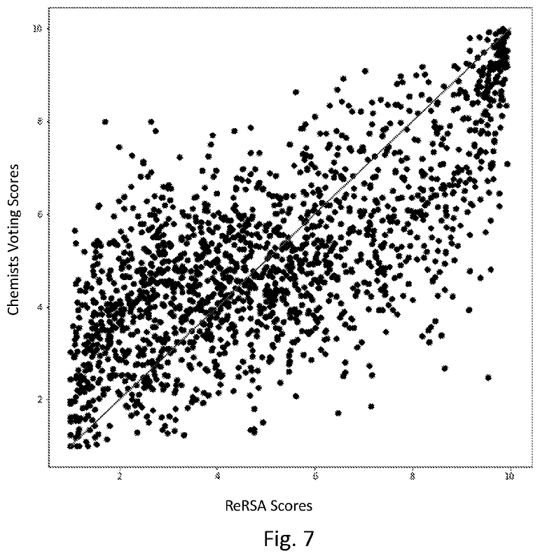

FIG. 5A illustrates a method 500 for training a model to calculate synthetic accessibility (SA). The method 500 can include receiving a training dataset of molecules to obtain the information of the chemical structure and other properties of one or more molecules (block 502). The method 500 then performs a protocol for decomposing molecules of (block 502) to sets of synthesizable fragments. The decomposition function should: produce valid drug-like molecular structures; and be invertible meaning that obtained fragments can be converted back to the original molecular structures. The method 500 includes evaluating fragments chemical properties (bock 506). The method 500 includes computing fragments frequencies among the training dataset (block 508). The method 500 includes computing fragment density function for the molecules in the training dataset (block 510). The method 500 includes aggregating obtained fragments information into fragments scores taking their frequencies into account (block 512). The method 500 includes providing a mechanism (e.g., computer and database) to store and obtain scores from block 512 (block 514). The method 500 includes calculating synthetic accessibility score (SAS) as a product between the fragment density function obtained at block 510 and a linear combination of the aggregated fragment information scores obtained at block 512 and the fragment frequencies database obtained at block 508 (block 516). In some embodiments, the training method includes normalizing the synthetic accessibility score to a desired scale with a mathematical function. In some embodiments, the training method includes normalizing the synthetic accessibility score to a desired scale with a mathematical function.

The method 500 can be performed with different variations. The receiving of the training dataset at block 502 can be performed by programmed tools. The decomposing into synthesizable fragments at block 504 can be performed by any retrosynthesis-related decomposing function, such as open-sourced BRICS or RECAP algorithms. The evaluation of fragment chemical properties at block 506 can be performed by calculation and aggregation of molecular and structural descriptors such as at least one of the following: Chiral Carbons Count=the number of chiral carbon atoms; Ring Count=the total number of rings; Ring Side Chains Count=the number of side chains attached to the ring systems; Spiro Count=the number of spiro carbon atoms; Biggest Ring Size=the number of atoms in the largest ring of molecular structure if it is bigger than 6, otherwise 0; Fused Rings Count=the number of fused rings in a molecular structure; and/or Bridge Atoms Count=the number of bridgehead atoms in the bicyclic pattern(s) of molecular structure. The computing of frequencies at block 508 is performed by applying a function, such as an identity or logarithm, to the number of molecules that contains a specific fragment divided by the number of molecules in the training dataset. The computing of fragment densities functions at block 510 is performed by applying a function, such as identity or linear function, to the number of atoms in the target molecule divided by the number of fragments in the target molecule. The aggregation of fragment information into a fragment score at block 512 is performed by any mathematical function applied to fragments descriptors and fragment frequencies. In some aspects, the input (e.g., training dataset of molecules) is presented by fragments.

FIG. 5B illustrates a method 550 for evaluating molecule synthetic accessibility. The method can include receiving a target molecule to obtain the information about its chemical structure and other related properties (block 552). The method 550 includes decomposing the received target molecule of block 552 to synthesizable fragments (block 554). The method 550 includes obtaining scores of synthesizable fragments (e.g., Fragment Scores, SD Score, etc.) from a trained model (block 556), such as a trained model obtained from the training methodology of FIG. 5A. The method 550 includes calculating molecular properties for fragments whose properties cannot be obtained in block 556 (block 558). The method 550 includes calculating fragment densities functions for fragments whose fragment densities functions cannot be obtained in block 556 (block 560). The method 550 includes aggregating processed information to obtain synthetic accessibility score of the target molecule (block 562). The method 550 can include obtaining and storing the synthetic accessibility score. In some aspects, the decomposing at block 554 is performed by any retrosynthesis-related decomposing function such as open-sourced BRICS or RECAP algorithms. In some aspects, the calculation of molecular properties in block 558 is performed by computing and aggregating chemical descriptors In some aspects, the calculation of fragment densities at block 560 is performed by computing fragments densities functions. In some aspects, the aggregation at block 562 is performed by mathematical formula applied to fragments scores. The some aspects the fragment scores (block 562) are scaled from one to n, where n>1. In some aspects, the vendor database is not present or used in the method 550 for evaluating molecule synthetic accessibility of a target molecule. In some embodiments, the training method includes normalizing the synthetic accessibility score to a desired scale with a mathematical function.

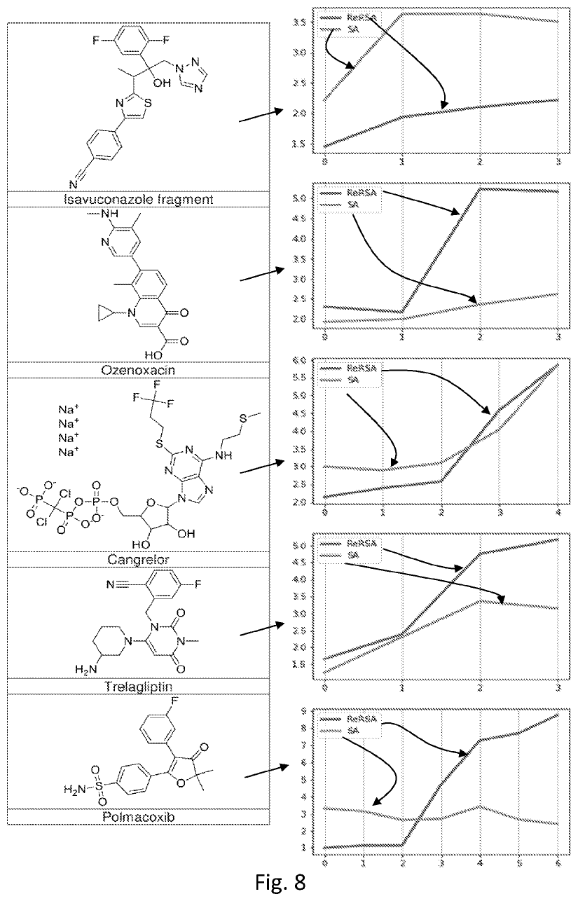

FIG. 6 shows a schematic representation of a computing device 600 (e.g., computer, cloud computing system, etc.) that can perform the computing methods described herein, which is described in more detail below.

The foregoing methods are described in more detail herein. During training, for obtaining “ready-to-synthesis fragments” from molecules, the ReRSA method uses a decomposition procedure that slices a target molecule into a set of fragments. Such a decomposition function should meet several key criteria. The first criterion is that each fragment has to be useful with bijective mapping, such that it should be possible to compose a molecule back given its obtained fragments. The second criterion is that any of the resulting fragments has to be an elementary building block, such that each fragment can be a part of a chemical reaction (reactants) to reach the target molecule. The latter also means that a RTSF is a valid molecular structure. An example of the decomposition function that meets all mentioned criteria is an open-sourced algorithm called BRICS or RECAP.

After each molecule in the training dataset is decomposed to synthesized fragments, the ReRSA protocol calculates and stores the frequencies of the synthesized fragments in a dictionary (e.g., database) over the whole dataset. Frequency of a fragment is the number of molecules from a prepared training dataset (e.g., in a database of molecules) containing the fragment, divided by the total number of molecules in the dataset. As a result, the frequency of a fragment will be always between zero and one, or it can be a percentage. Therefore, if the frequency of a fragment is low (e.g., below a frequency lower bound threshold) it will not contribute much to the synthetic accessibility score (SAS) of the method and vice versa. In other words, rarely synthesized fragments are usually harder to synthesize than frequently synthesized fragments. While frequencies of fragments can be used as is, the approach takes a minus logarithm of it, so it makes a bigger contribution to overall score. See:

fr_{frag}=1−log(frequency)

There are several variants how can fragment frequency be defined:

fr_{frag}=1−frequency,

fr_{frag}=termfrequency(fragment) is frequency of fragment in fragments space.

Then ReRSA computes an intermediate synthetic difficulty (SD) score (SD Score) of each RTSF in a molecule taking into consideration the fragment's precalculated frequency value. Intuitively, the SD Score represents chemical complexity of the fragment in terms of its usage in the training dataset and its biochemical properties. The SD Score (also referred to herein as sd) is based on carefully selected and well-tuned molecular descriptors (MD) and is defined as follows:

sd=(ChiralCarbonsCount+RingCount+RingSideChainsCount+SpiroCount+BiggestRingSize+FusedRingCount+BridgeAtomsCount)·Q1

Formula of sd includes several listed molecular descriptors:

Chiral Carbons Count is the number of chiral carbon atoms;

Ring Count is the total number of rings;

Ring Side Chains Count is the number of side chains attached to the ring systems;

Spiro Count is the number of Spiro carbon atoms;

Biggest Ring Size is the number of atoms in the largest ring of molecular structure if it is bigger than 6, otherwise 0,

Fused Rings Count is the number of fused rings in a molecular structure;

Bridge Atoms Count is the number of bridgehead atoms in the bicyclic pattern(s) of molecular structure; and

Q1 is normalized quadratic index 1 calculated as (3−2*A+Z1/2), where A is the number of heavy atoms, and Z1 is the first Zagreb index.

All MDs in the formulas of SD Score have a strong chemical relevance and highly correlate with the complexity of the fragment meaning that from a chemical point of view the increase in any MD of the fragment should definitely increase its entanglement and complexity.

However, the presented SD Score can have one potential problem. Some molecules can be too complex meaning that they cannot be split in a set of fragments. This implies that the SD Score can be lower for such molecules than it should be. To cope with this problem the ReRSA method introduces a special hyperparameter called fragment density (FD). The FD measures a relative density of synthesizable fragments that can be found in a molecule. In the simplest case it can be defined as a number of atoms divided by the number of synthesizable fragments in a molecule. It is also clear that the simplest case of FD increases with increasing of the number of atoms and decreases with the increasing of the number of fragments. So, FD will increase the total score for molecules with less amount of fragments. However, the hyperparameter can be designed in a more principal way. For instance, it can take into account not a single molecule with its atoms and fragments but a set of neighborhood molecules with respect to a target one by some similarity metric and thus aggregates topological information about the neighbor molecules.

The last stage of the ReRSA method is the calculation of the final score called ReRSA Score which corresponds to synthetic accessibility score (SAS) of a whole molecule. The unnormalized version of ReRSA Score is defined as a product between FD and the sum of SD Scores of all synthesizable fragments that are found in a target molecule weighed by their computed frequencies as follows:

$${Re RSA}_{unnorm} = ( {\sum }_{frag ∈ fragments} {sd}_{frag} \cdot {fr}_{frag} ) \cdot FD$$

It can be seen from the formula above that the final score can take values from zero to infinity, so it is not normalized. To make the ReRSA score more user-friendly and meaningful in terms of medicinal chemistry one or more normalizing functions can be employed. For instance if the desired value of the score should be between zero and one then sigmoid function can be used. To achieve the score in a specific predefined diapason a method can, for example, apply arctangent function with some range specific parameters. In the case of arctangent the ReRSA Score is defined as:

$$ReRSA = arc tan ( \frac{{ReRSA}_{unnorm}}{SC} ) \cdot \frac{2}{π} \cdot UL + 1$$

Here, SC is the scale hyperparameter and UL is the upper limit of the ReRSA score. The goal of SC is to provide better distinction between parts of molecules space. Lower SC leads to decrease of scores, while bigger SC leads to opposite. The correct choice of SC must result in smooth and centered distribution of ReRSA scores. The SC equal to ten thousand was chosen according to the results of experiments. There is a production standard, that requires a scaling score from one to ten, which provided by UL equal to nine.

It should be emphasized that the ReRSA method is very different compared to SA Score (SAS). The SA Score uses molecular descriptors computed on fragments obtained from most frequent training fingerprints (precisely on extended connectivity fingerprints), which are not necessarily valid, especially synthesizable molecular structures. Such fingerprints are not appealing in terms of medicinal chemistry and cannot be used as building blocks to provide rational chemical synthesis planning. Furthermore, ReRSA takes into account much more chemically relevant molecular descriptors than SA Score.

Another aspect is that the choice of training dataset is very important because it directly affects the frequencies of fragments, and thus contributes much to the overall ReRSA score. The processes of collecting, preprocessing such a training dataset are further elaborated in the text.

The ReRSA method is wholly developed in the Python programming language. Decomposition procedure as well as all molecular descriptors are implemented and calculated using the RDKit library. Graphics are drawn with matplotlib library.

The training algorithm of the ReRSA method is shown below:
1. Create a dictionary in which information about synthesizable fragments will be stored,
2. Split every molecule in synthesizable fragments and store them in a list without preserving identical synthesizable fragments within the same molecule,
3. Calculate frequencies:
   a) Count every unique synthesizable fragment occurrences in the fragments list,
   b) Divide that count by number of the molecules in the training dataset,
4. Calculate molecular descriptors for every unique fragment,
5. Aggregate descriptors and frequencies into sd for every fragment.

A fragmentation algorithm can be used with a Vendor molecule database M of size m, and with a Dictionary of fragment Frequencies D_{fr}, and a Dictionary of fragment sd D_{sd}:

Algorithm 1: Training Procedure of the SA predictor

| | |
|---|---|
| 1. | for “m”-steps do: |
| 2. | split molecule into fragments F = (f₁, . . ., Fₙ) |
| 3. | for f in 1; N do |
| 4. | D_{fr} [f] = D_{fr}[f] +1/m |
| 5. | end for |
| 6. | end for |
| 7. | Fr = keys of D_{rf}; |
| 8. | K = length of Fᵣ; |
| 9. | for k ∈ 1; K do |
| 10. | Compute descriptors (Chiral Central Count, Ring Count, |
| | Ring Side Chain Count, Spiro Count, Biggest Ring Size, |
| | Fuse Rings Count, Bridge Atoms Count, Q1). |
| | D_{sd}[Fᵣ[k]] = Chiral Center Count + Ring Count + Ring Side |
| | Chain Count + Spiro Count + Biggest Ring Size + Fused |
| | Ring Count + Bridge Atoms Count) · Q1 · (1−D_{fr}[Fᵣ[k]]) |
| 11. | end for |

Once the ReRSA is trained its score can be achieved by the following scheme:
1. Receive a new molecule,
2. Split molecule into synthesizable fragments,
3. For every synthesizable fragment:
   If synthesizable fragment is present in train sample, we take calculated sd,
   Else MDs are calculated and imply that frequency equals

$$\frac{1}{length ( training dataset )} ,$$

4. Calculate FD as

$$\frac{n umber of atoms}{n umber of fragments} ,$$

5. Aggregate sd and FD into ReRSA score.

A fragmentation algorithm can be used as SA predictor, with a Dictionary of a dictionary of fragment sd D_{sd}; Molecule M; Scaling parameters SC; and Upper limit parameter UP:

Algorithm 2: Scoring procedure of the SA predictor

| | |
|---|---|
| 1. | split molecule into fragments F = (f₁, . . ., Fₙ) |
| 2. | SA = 0 |
| 3. | for n ∈ 1; N do |
| 4. | SA + SA + D_{sd}[fₙ] |
| 5. | end for |
| 6. | Nₐ = number of atoms in M |
| 7. | D = Nₐ/N |
| 8. | SA = arctan ((SA·D)/SC) · UP + 1 |

In another option, once the ReRSA is trained its score can be achieved by the following scheme:
1. Receive a new molecule,
2. Split molecule into synthesizable fragments,
3. For every synthesizable fragment:
   If synthesizable fragment is present in train sample, we take calculated sd,
   Else MDs are calculated and imply that frequency equals:

fr_{frag}=1−log(frequency)
4. Calculate FD as:

fr_{frag}=1−frequency
5. Aggregate sd and FD into ReRSA score.

A fragmentation algorithm can be used as SA predictor, with a Dictionary of a dictionary of fragment sd's D_{sd}; Molecule M; Scaling parameters SC; and Upper limit parameter UP:

Algorithm 2: Scoring procedure of the SA predictor

| | |
|---|---|
| 1. | split molecule into fragments F = (f₁, . . ., Fₙ) |
| 2. | ReRSA = 0 |
| 3. | for n ∈ 1; N do |
| 4. | ReRSA + ReRSA + D_{sd}[fₙ] |
| 5. | end for |
| 6. | Nₐ = number of atoms in M |
| 7. | D = Nₐ/N |
| 8. | ReRSA = normalize ((ReRSA·D)/SC) |

### Examples

Validation

In some embodiments, the SA is a very subjective term and every BigPharma or biotech company defines SA in their own manner. Thus, several distinct experiments are conducted to objectively compare the ReRSA method to the well-known SA Score.

As a training dataset for all of the experiments ZINC15 was used. It consists of ˜230M available in stock chemicals. The dataset was pre-processed according to the following procedure:
1. The compounds with molecular weights greater than 1000 Da were removed from the dataset.
2. Salt parts were removed from the records. The resulting duplicates were then removed.
3. The metal-containing chemicals were removed.
4. Advanced in-house medicinal chemistry filters (e.g. PAINS substructures and toxicophores) were applied in order to filter the dataset from non-relevant compounds. Nature-like compounds (e.g. steroids, flavonoids, (oligo)sugars, (oligo)peptides etc) were removed from the dataset as they are not related to a pure synthetic chemistry.
5. The resulting dataset of ˜7M compounds was clusterized into the clusters with minimum Tanimoto similarity 0.5 and singletons were adjusted to the nearest clusters. Then 1% of diverse molecules were extracted from each cluster and the resulting dataset contained ˜1.2M compounds that describes chemical space of synthetic compounds interesting from a medicinal chemistry perspective.

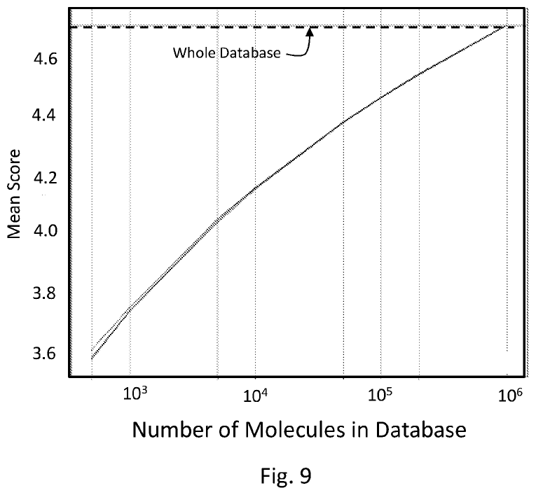

To determine whether or not ReRSA Scores are meaningful in terms of medicinal chemistry, a first experiment for the correlation between the ReRSA Score and medicinal chemists estimates is performed. For that purpose the dataset and chemist scores of synthetic accessibility were collected (pubs.acs.org/doi/10.1021/ci5001778) and then ReRSA Scores were calculated. As a result, the method achieves a Pearson correlation coefficient of 0.702 (p-value=1.035e-257) with respect to chemists' scores. FIG. 7 shows dependency between two scoring engines.

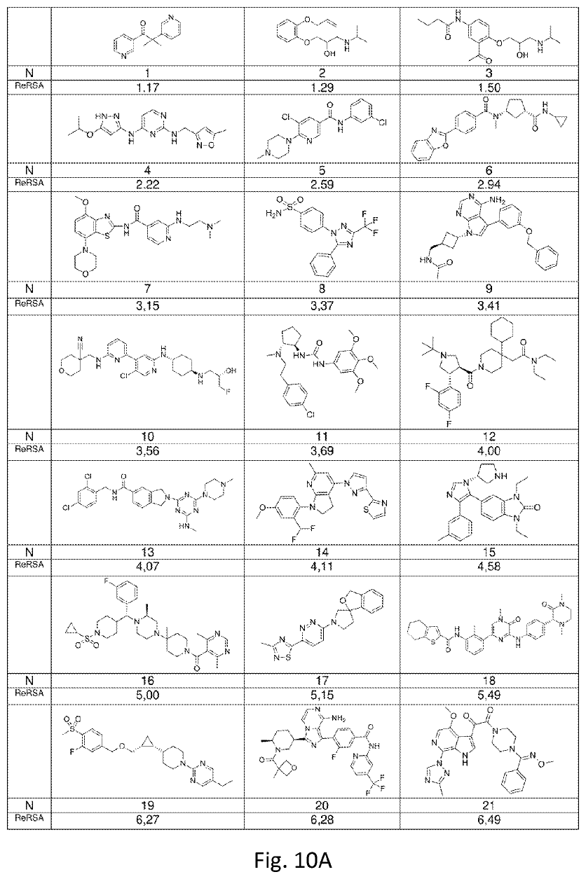

The second experiment is performed to the evaluation of the ReRSA method in the case of retrosynthesis. Five well-known compounds and their retrosynthetic routes are selected and then for each step in every synthetic route two scores are computed: the ReRSA Score and SA Score. FIG. 8 shows the dependency between the scores and the steps in the selected routes.

Because all routes do not have protection/deprotection steps the utopian score should behave as a monotonically increasing function. It is clearly seen from the figures that ReRSA Score is better in terms of monotonicity than SA Score.

The third experiment relates to the consistency of the training dataset as well as answers a question about what the optimal size of the training dataset should look like. Firstly, to estimate the consistency of the training dataset it is split half by half and ReRSA Score is calculated for both parts of the original training dataset. The achieved Pearson correlation between those parts is 0.99 meaning that the dataset is unbiased and represents enough synthesizable fragments for the training of the method. In some aspects, the training dataset is split in the batches.

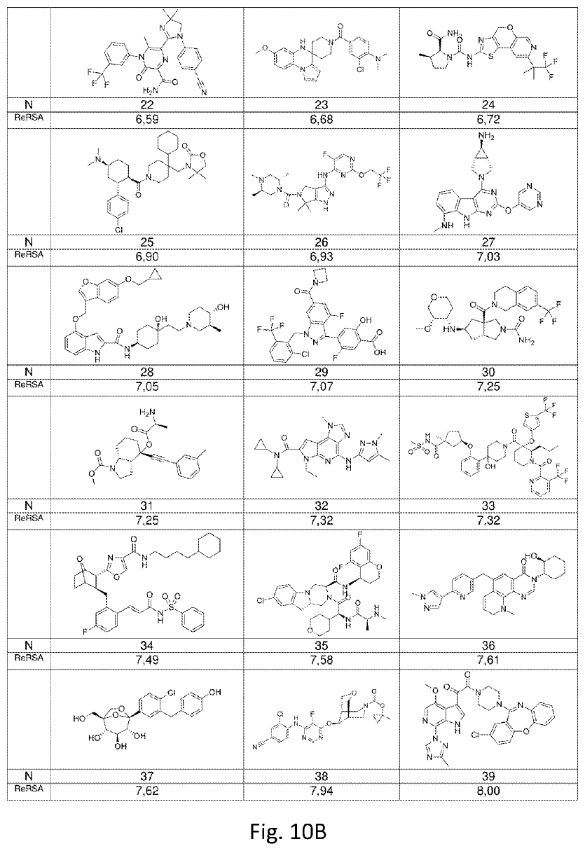

Experiments can determine how the predictor depends on the size of the database. The graph in FIG. 9 shows the dependence of scores on the size of the training dataset. Initial base was shuffled three times and then parts of it were used for learning. All sizes of the parts are cumulative within one attempt: bigger databases contain every molecule from smaller ones. Evaluation was performed on a batch of 1000 molecules not represented in the initial database.

It can be seen that mean scores does not change much from launch to launch, which means algorithm are robust to sampling from database. Although the scores tend to increase with dataset size, which is obvious because frequencies cannot increase with the addition of the new fragments. One can also notice that the mean scores are pretty close to red line even at a hundred thousand samples, which is less than ten percent of the whole dataset. See FIG. 9.

In order to establish the scaling and threshold for the scoring function output the following experiment was carried out. From the organic synthesis expertise, the scale from 1 to 10 of ReRSA scoring based on the training dataset discussed above should be divided into 5 ranges:
1-2—very easy to make compounds. Usually includes the compounds that are being splitted into 2-4 very common building blocks (BBs).
2-4—easy to make compounds. Usually the molecules that can be constructed from 3-6 building blocks and using common organic synthesis reactions. Even large compounds (500-700) can have ReRSA in this range if they could be completely fragmented into the common building blocks. Usually the synthesis for compounds in this range requires 4-8 easy-to-perform steps.
4-6—Commonly 4-10 routes steps are required to synthesize the molecules from this ReRSA range. Many of the compounds are presented in the medicinal chemistry outputs from BigPharma companies in the last decade. This range is the “golden mean” of the scale. We recommend taking into account first the compounds from this range as they share equally good complexity and synthetic accessibility.
6-8—Challenging but quite possible-to-synthesize compounds. Many of the compounds are presented in the medicinal chemistry outputs from BigPharma companies in the last decade. Many of the compounds require 6-12 stages using purchasable BBs. Chemists may struggle with the synthesis of molecules in 7-8 range.
8-10—Very challenging molecular structures. Multistep (more than 12-15 stages) synthesis is required (8-9) or almost impossible (9-10) to synthesize using common techniques. Sophisticated macrocycles, nature-like compounds, compounds containing rare polycondensed heterocycles and plenty of stereocenters are predominantly scored in this range. 9-10 usually requires a very sophisticated retrosynthesis route.

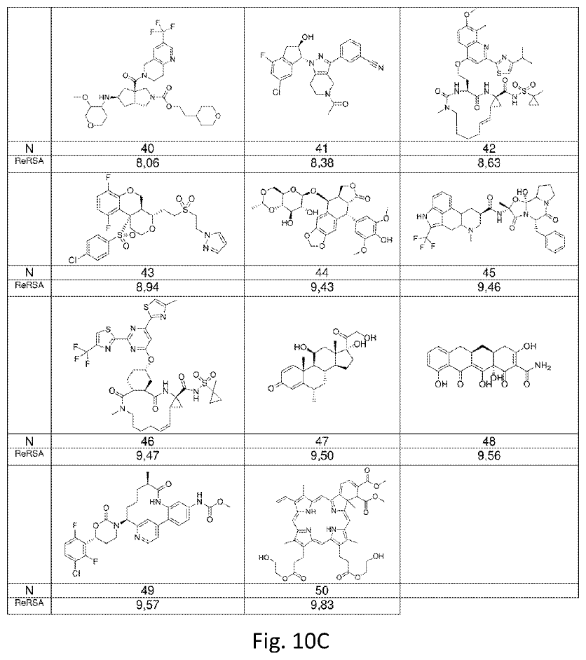

The value of 8 is recommended as a default threshold and 8.5 as a mild threshold. In the table of FIGS. 10A-10C representative examples of known bioactive compounds accompanied by the calculated ReRSA Scores are listed. The tables of FIG. 10A-10C are arranged in the ReRSA Score increasing order, with those in FIG. 10B increasing from those in FIG. 10A. and those in FIG. 10C increasing from those in FIG. 10B.

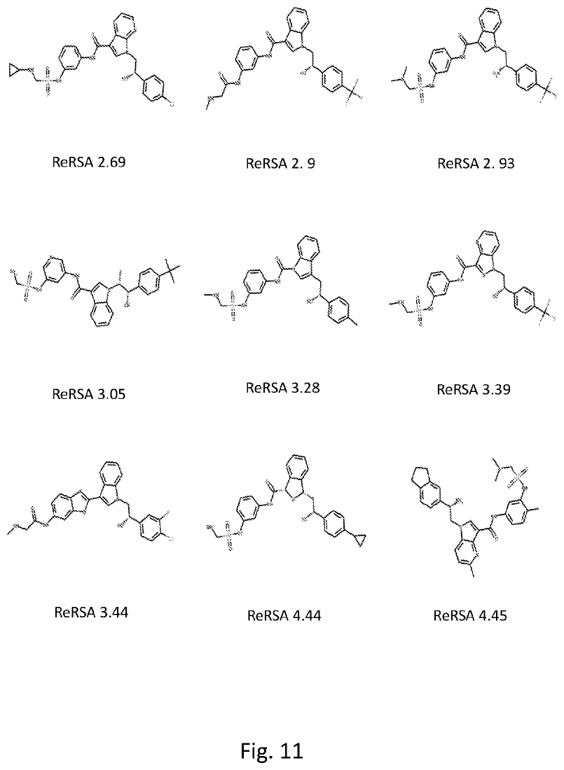

The experiment 5 was carried out on the set of similar compounds with small variations in their structure in order to show that ReRSA score is sensitive to these small variations (e.g. insertion or deletion of 1. One or two heteroatoms into the cycles, 2. Extra chiral carbon, 3. Csp2 (Aro)-Csp2(Aro) bond pattern etc) as described in the figure below and the appearance of hard-to-synthesize patterns leads to the increase of ReRSA Score. That means that ReRSA Score appears to be useful from organic and medicinal chemistry perspective in the high-throughput prioritization of molecular structures for their synthetic accessibility rapid estimation and submission for further synthesis. See FIG. 11.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In one embodiment, the present methods can include aspects performed on a computing system. As such, the computing system can include a memory device that has the computer-executable instructions for performing the method. The computer-executable instructions can be part of a computer program product that includes one or more algorithms for performing any of the methods of any of the claims.

In one embodiment, any of the operations, processes, methods, or steps described herein can be implemented as computer-readable instructions stored on a computer-readable medium. The computer-readable instructions can be executed by a processor of a wide range of computing systems from desktop computing systems, portable computing systems, tablet computing systems, hand-held computing systems as well as network elements, base stations, femtocells, and/or any other computing device.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

FIG. 6 shows an example computing device 600 that is arranged to perform any of the computing methods described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including but not limited to a microprocessor (μP), a microcontroller (μC), a digital signal processor (DSP), or any combination thereof. Processor 604 may include one more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations memory controller 618 may be an internal part of processor 604.

Depending on the desired configuration, system memory 606 may be of any type including but not limited to volatile memory (such as RAM), non-volatile memory (such as ROM, flash memory, etc.) or any combination thereof. System memory 606 may include an operating system 620, one or more applications 622, and program data 624. Application 622 may include a determination application 626 that is arranged to perform the functions as described herein including those described with respect to methods described herein. Program Data 624 may include determination information 628 that may be useful for analyzing the contamination characteristics provided by the sensor unit 240. In some embodiments, application 622 may be arranged to operate with program data 624 on operating system 620 such that the work performed by untrusted computing nodes can be verified as described herein. This described basic configuration 602 is illustrated in FIG. 6 by those components within the inner dashed line.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, the term “module” or “component” can refer to software objects or routines that execute on the computing system. The different components, modules, engines, and services described herein may be implemented as objects or processes that execute on the computing system (e.g., as separate threads). While the system and methods described herein are preferably implemented in software, implementations in hardware or a combination of software and hardware are also possible and contemplated. In this description, a “computing entity” may be any computing system as previously defined herein, or any module or combination of modulates running on a computing system.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

1. A method for training a model to calculate synthetic accessibility, comprising:
accessing a molecule database and obtaining a target molecule;
slicing the target molecule into molecular fragments;
determining a fragment frequency of a plurality of molecular fragments of the target molecule;
calculating molecular descriptors for the molecular fragments;
calculating a synthetic difficulty score for the target molecule; and
storing the synthetic difficulty score for the target molecule in a database having a plurality of synthetic difficulty scores for a plurality of molecules.

2. The method of claim 1, comprising receiving a training dataset of training molecules to obtain data of a chemical structure and properties of the target molecule.

3. The method of claim 1, the slicing comprising decomposing the target molecule to obtain synthesizable fragments, where a decomposition function:
produces valid drug-like molecular structures; and
is invertible so that obtained synthesizable fragments can be converted back to the target molecule.

4. The method of claim 3, wherein the decomposing is performed by a retrosynthesis-related decomposing function.

5. The method of claim 1, comprising evaluating chemical properties of the synthesizable fragments.

6. The method of claim 5, wherein the evaluating is performed by calculation and aggregation of the molecular descriptors.

7. The method of claim 6, wherein the aggregation of molecular descriptors includes:
Chiral Carbons Count, which is the number of chiral carbon atoms;
Ring Count, which is the total number of rings;
Ring Side Chains Count, which is the number of side chains attached to the ring systems;
Spiro Count, which is the number of spiro carbon atoms;
Biggest Ring Size, which is the number of atoms in the largest ring of molecular structure if it is bigger than 6, otherwise 0;
Fused Rings Count, is the number of fused rings in a molecular structure; and
Bridge Atoms Count, is the number of bridgehead atoms in the bicyclic pattern(s) of molecular structure.

8. The method of claim 2, wherein determining the fragment frequency is performed by applying a function of identity or logarithm to the number of molecules that contain the molecular fragment divided by the number of molecules in the training dataset.

9. The method of claim 2, comprising computing a fragment density function for the target molecule across the training dataset of training molecules based on the frequencies of the synthesizable fragments in the training molecules.

10. The method of claim 2, comprising aggregating fragment information of synthesizable fragments of the target molecule into fragment scores by taking the fragment frequencies into account.

11. The method of claim 10, wherein the aggregating is performed by a mathematical function applied to molecular descriptors of fragments and fragment frequencies.

12. The method of claim 10, comprising obtaining the fragment scores and saving the fragment scores in a database of fragment scores.

13. The method of claim 10, comprising calculating a synthetic accessibility score as a product between a fragment density function and a linear combination of fragment scores and fragment frequencies.

14. The method of claim 13, comprising at least one of:
providing the calculated synthetic accessibility score; or
normalizing the calculated synthetic accessibility score to a scale by a mathematical function.

15. A method of evaluating molecular synthetic accessibility, the method comprising:
selecting a target molecule;
decomposing the target molecule into molecular fragments;
calculating a synthetic difficulty score for the molecular fragments for the target molecule;
determining a sum of synthetic difficulty scores for the molecular fragments;
determining a fragment density of the molecular fragments;
calculating the synthetic accessibility score from the sum of synthetic difficulty scores and fragment densities; and
providing the synthetic accessibility score for the target molecule.

16. The method of claim 15, comprising obtaining data of chemical structure and properties of the target molecule.

17. The method of claim 15, comprising obtaining scores of synthesizable fragments from a trained model for calculating synthetic accessibility.

18. The method of claim 17, comprising calculating molecular properties for fragments whose properties cannot be obtained from the trained model.

19. The method of claim 18, comprising calculating fragment density functions for fragments whose fragment density functions cannot be obtained from the trained model.

20. The method of claim 15, comprising aggregating processed information to the synthetic accessibility score of the target molecule.

21. The method of claim 15, wherein the decomposing is performed by a retrosynthesis-related decomposing function, optionally selected from open-sourced BRICS or RECAP algorithms.

22. The method of claim 15, comprising evaluating chemical properties of the synthesizable fragments.

23. The method of claim 22, wherein the evaluating is performed by calculation and aggregation of the molecular descriptors.

24. The method of claim 23, wherein the aggregation of molecular descriptors includes:
Chiral Carbons Count, which is the number of chiral carbon atoms;
Ring Count, which is the total number of rings;
Ring Side Chains Count, which is the number of side chains attached to the ring systems;
Spiro Count, which is the number of Spiro carbon atoms;
Biggest Ring Size, which is the number of atoms in the largest ring of molecular structure if it is bigger than 6, otherwise 0;
Fused Rings Count, is the number of fused rings in a molecular structure; and
Bridge Atoms Count, is the number of bridgehead atoms in the bicyclic pattern(s) of molecular structure.

25. The method of claim 15, comprising computing a fragment density function for the target molecule across the training dataset of training molecules based on the frequencies of the synthesizable fragments in the training molecules.

26. The method of claim 15, comprising aggregating processed information of synthesizable fragments of the target molecule into fragment scores by taking the fragment frequencies into account.

27. The method of claim 26, wherein the aggregating is performed by a mathematical function applied to molecular descriptors of fragments and fragment frequencies.

28. The method of claim 15, wherein the synthetic accessibility score are scaled from one to n, where n>1.

29. The method of claim 15, wherein a vendor database for the target molecule or synthesizable fragments is not present.

30. The method of claim 15, comprising:
calculating a synthetic difficulty score for the target molecule by an iterative protocol including:
identifying all molecular fragments of the target molecule;
checking for all molecular fragments in a synthetic difficulty score database;
when a molecular fragment is the synthetic difficulty score database, add the synthetic difficulty score for the molecular fragment to an array of synthetic difficulty scores;
when a molecular fragment is not in the synthetic difficulty score, then:
calculate molecular descriptor for the molecular fragment;
calculate the synthetic difficulty score for the fragment with a minimum frequency; and
add the calculated synthetic difficulty score for the molecular fragment to an array of synthetic difficulty scores.

31. One or more non-transitory computer readable media storing instructions that in response to being executed by one or more processors, cause a computer system to perform operations, the operations comprising the computer method of claim 1.

32. One or more non-transitory computer readable media storing instructions that in response to being executed by one or more processors, cause a computer system to perform operations, the operations comprising the computer method of claim 15.

33. A computer system comprising:
one or more processors; and
one or more non-transitory computer readable media storing instructions that in response to being executed by the one or more processors, cause the computer system to perform operations, the operations comprising the computer method of claim 1.

34. A computer system comprising:
one or more processors; and
one or more non-transitory computer readable media storing instructions that in response to being executed by the one or more processors, cause the computer system to perform operations, the operations comprising the computer method of claim 15.
